(19) United States
(12) Patent Application Publication
(10) Pub. No.: US 2005/0085876 A1
(43) Pub. Date: Apr. 21, 2005

(54) Solarium

(76) Inventor: Scalco Alfredo, Bassano Del Grappa (IT)

Correspondence Address: BUCKNAM AND ARCHER, 1077 Northern Boulevard, Roslyn, NY, 11576-1696, US

(21) Appl. No.: 10/686,364

(22) Filed: Oct. 15, 2003

(51) Int. Cl. A61N 5/06

(52) U.S. Cl. 607/94

(57) ABSTRACT

The solarium (1) comprises a support (2) on which press a frame (3) which supports a bed (4). The bed forms the surface (5) for resting on which the user may rest. The cover (7) is disposed above the bed and means to generate ultraviolet rays (8) which are disposed in the bed and in the cover. The frame (3) is connected to the support (2) by connection means (11) which form an horizontal axis of rotation (12). The bed (4) and the cover (7) rotate around the axis (12) to define different positions of use.

### BACKGROUND OF THE INVENTION

1. Field of the Invention

This invention relates to a solarium for tanning treatments particularly suitable to be configured to function in different positions for the purpose of satisfying the requirements of the user.

2. Background of the Prior Art

It is known that there are some specialized aesthetic centers in which it is possible to undergo tanning treatments. These treatments essentially provide a display which is controlled with tanning rays which are, in general, constituted by UV radiations.

The solariums of known type differ among themselves for the type of ultraviolet radiations being used for the typology of the sources and for the functional and structural characteristics of the solarium itself.

More accurately, we find among the solariums of known type, the sunbed solarium and the shower bath type solarium. The sunbed solarium comprises, essentially, a basement on which is pressed a frame which supports a bed, the latter forming a shaped surface for resting on which the user is extended during the treatment.

On top of the bed there is a cover, which during the treatment, covers the user and limits or defines an essentially closed volume. More accurately, the cover is movable from a position essentially parallel to the bed to an essentially orthogonal position which is necessary to allow access for the user.

Suitable lamps equipped with special reflecting structures and located in the interior of the bed and/or of the cover generate ultraviolet radiations which are necessary to obtain the desired tanning.

In particular, the lamps are supplied with power controlled by means of a command console which allows the ignition and the extinguishing as a function of the desired treatment.

With respect to the shower bath type solarium, this one is different from the solarium described herein above due to the fact that the user during the treatment stands up. More accurately, the shower bath type solarium consists essentially of a vertical hollow frame composed of two parts connected between themselves to allow access for the user.

The choice between the sunbed solarium and the shower bath type solarium is carried out by the user on the basis of his requirements and his preferences.

On the basis of the descriptions herein above, it is clear that an aesthetic center in order to comply with the requirements of the client must be provided with both types of solarium, with obvious costs of purchasing, arrangement and maintenance.

### SUMMARY OF THE INVENTION

An object of the present invention is to provide a multifunction solarium which may be configured to function both as a sunbed and a shower bath type solarium.

Another object of this invention is to provide a solarium which allows the operator to select the areas to treat as a function of the requirements of the user.

Still, another object is to provide a solarium which allows to locate the area of treatment in anyone of the points of interest of the user.

Still, another object of this invention is to achieve a solarium simple in use and reliable.

The mentioned objects are achieved by a solarium which comprises a support on which a frame is pressed, the frame supporting at least a bed, the latter forming a shaped surface for resting the user and at least a cover located above the bed to cover the user. Means to generate ultraviolet rays are disposed on the support in the bed, they are in the cover and are supplied with a power group.

The frame is characterized by the fact that it is connected to the support by connection means which form at least one case of essentially horizontal rotation around which the bed and the cover rotate to define different positions of use.

Advantageously, the solarium object of this invention may be configured in a manner to adapt itself to the specific requirements of the user because it may be disposed both as a bed and as a shower bath type solarium.

### BRIEF DESCRIPTION OF THE DRAWINGS

The objects and the advantages will be better appreciated by reference to a preferred not limiting execution offered for clarification by reference to the drawings, of which:

FIG. 1 shows a side view of the solarium of this invention in a first configuration of use;

FIG. 2 shows another side view of the solarium of FIG. 1;

FIG. 3 shows a side view of the solarium of FIG. 1 and shows at least some of the internal structures;

FIG. 4 shows a side view of another configuration of use of the solarium of FIG. 1;

FIG. 5 shows a side view of a further configuration of use of the solarium of FIG. 1;

FIG. 6 shows a side view of one of the possible configurations of use of the solarium of FIG. 1.

### DETAILED DESCRIPTION OF THE INVENTION

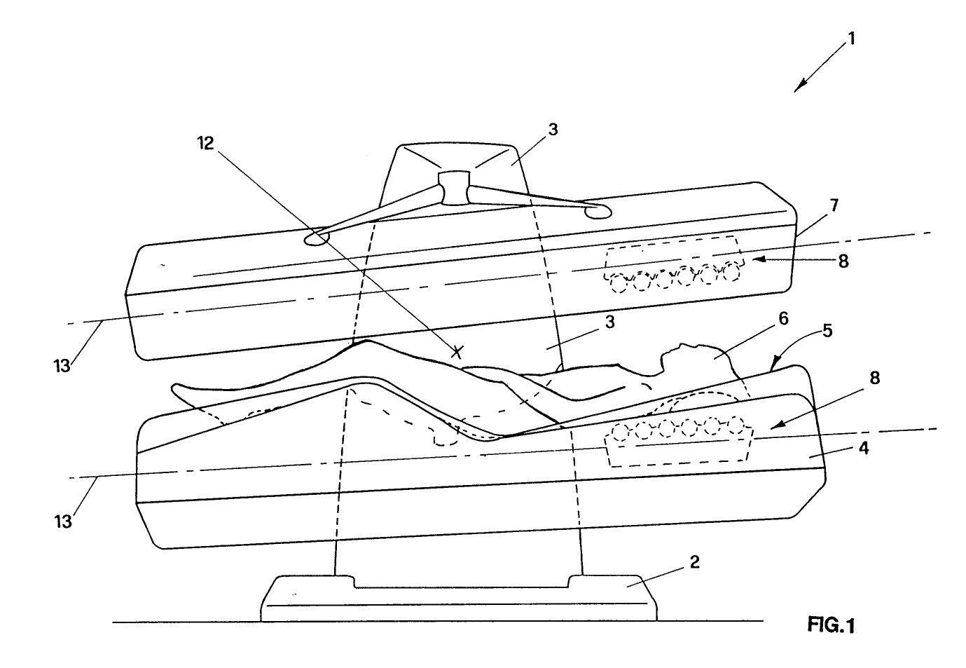

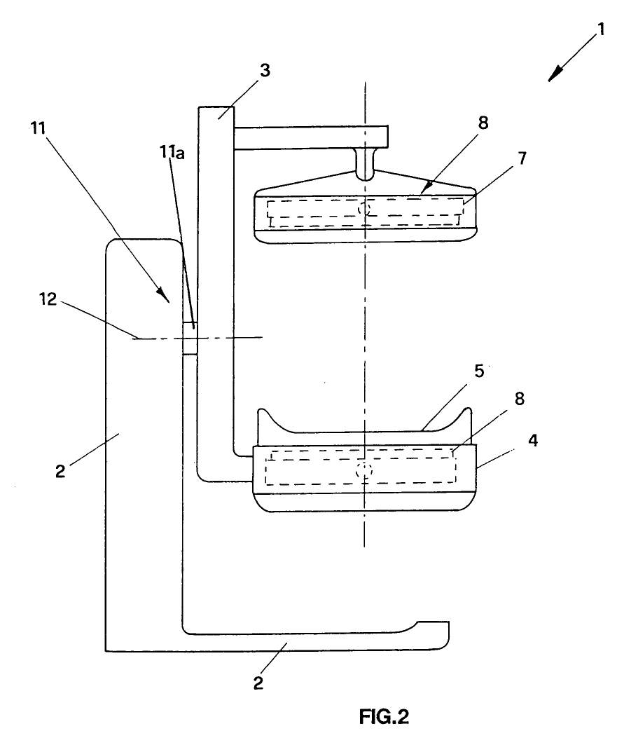

The solarium, which is the object of this invention is shown in FIG. 1 in which it is overall designated by numeral 1. The solarium comprises support 2 on which presses frame 3 visible in detail in FIG. 2. This frame supports bed 4. The bed forms a transparent shaped surface 5 of rest on which the user 6 extends himself.

Cover 7 is disposed above the bed to cover the user 6 during the treatment, and limits the substantially closed volume in which is disposed the user 6.

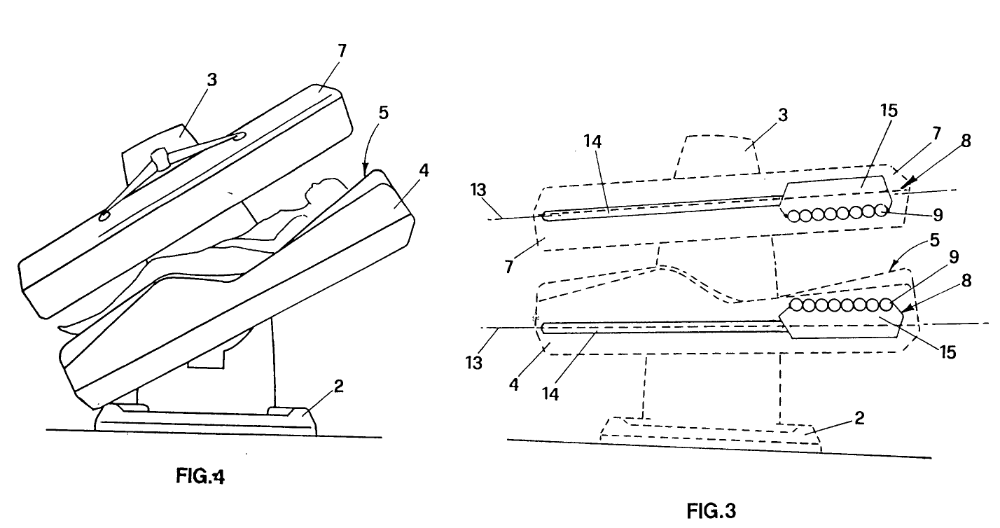

Means which generate ultraviolet rays are designated overall by reference numeral 8. They are constituted in the example by lamps with metal iodide 9 visible in detail in FIG. 3. They are disposed in the bed 4, under the shaped surface 5 and in cover 7. In particular, the lamps with metal iodide 9 are electrically connected to a power group, not shown, which feeds them supplying the energy necessary to irradiate the user 6 with the UV rays.

This invention provides that the frame 3 be connected to support 2 by means of connection means visible in FIG. 2 designated overall by numeral 11 which form an axis of rotation 12 substantially horizontal around which the bed 4 and the cover 7 rotate to define different positions of use shown in particular in FIGS. 1, 4 and 5.

More accurately, the connection means 11 are constituted in the example by a tree 11a which is integral with frame 3 and which forms the axis of rotation 12. They cooperate in addition with the first handling means capable of moving the bed 4 and the cover 7 around the axis 12 up to reaching different positions of use.

These first handling means are constituted by an electric motor which is provided with Kinematic means connected mechanically to tree 11a which forms the axis of rotation 12.

As far as it is related to the metal iodide lamps 9, they are supported by guide means visible in FIG. 3 which are associated with bed 4 and cover 7.

The guide means form a shifting direction designated by numeral 13, parallel to the longitudinal axis which is formed by bed 4 and cover 7 along which lamps 9 move.

In particular, the guide means are constituted by a rail 14 to which is coupled carriage 15 in a smooth flowing manner, the carriage supporting lamps 9. The carriage is moved by means of second handling means preferably constituted by an electric motor provided with Kinematic means of the type which is known so that it is not described.

A first variation in the execution, which has not been represented, differs from the described embodiment due to the fact that the lamps are fixed and distributed uniformly along longitudinal directions parallel to the axis of the bed 4 and cover 7.

As far as it is related to the power group, this comprises means of interruption of the electric feeding to lamps 9, these means being capable of turning on and turning off the lamps 9 to activate them selectively for the purpose of varying the areas of display to UV rays.

A control unit provided with interface means constituted in general by a keyboard and cooperating with the power group and with the handling means allows the operator to control and vary the configuration of work of the solarium.

In operation, a session of treatment foresees that the operator, after hearing the requirements and the preferences of the individual who will undergo the treatment, inserts the data necessary for the control unit by means of the keyboard.

Automatically, after these data are confirmed, the solarium will be disposed in the chosen configuration, for instance, taking an arrangement as a small bed.

Subsequently, the operator always by means of the control unit, lifts the cover 7, as shown in FIG. 6, allowing the user 6 to lay down.

Finally, the cover 7 is lowered and the lamps 9 are turned on so that the solarium is brought in the configuration shown in FIG. 1 and initiating the treatment which will provide the motion of lamps 9.

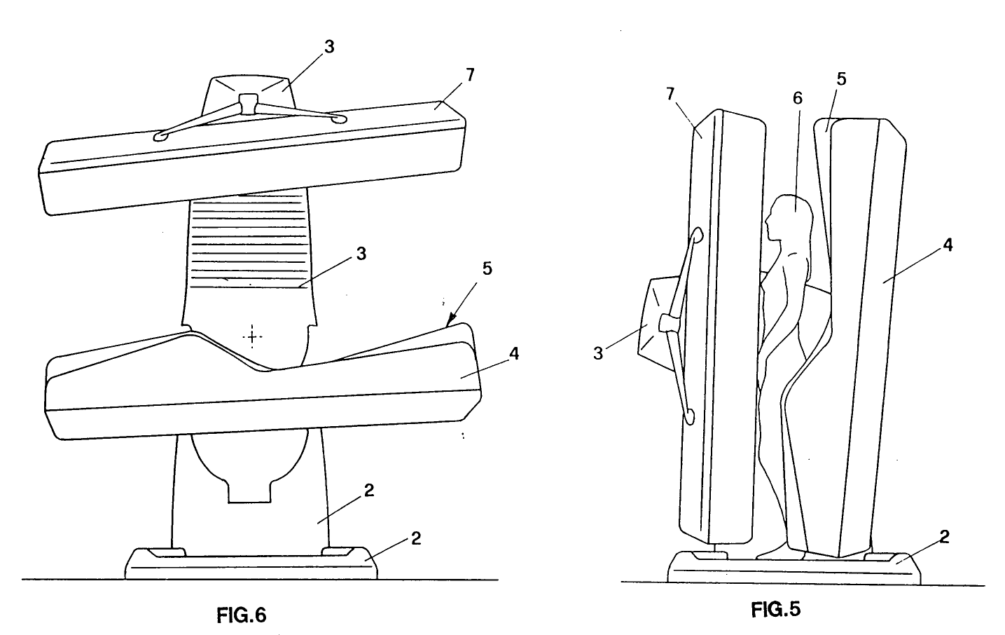

In the alternative, the solarium on the basis of the requirements of the user, may be disposed for instance in the shower bath type configuration as shown in FIG. 5.

In analogy with the preceding case, the operator acting on the control unit puts the cover further out to allow the user 6 to stand up in the interior of the volume formed by the bed 4 and cover 7 closed during the treatment.

The solarium, therefore, may be disposed in different positions, intermediary to the described position as shown for instance in FIG. 4 for the purpose of complying with the requirements of the client and improve the requested treatment.

It is important, in addition, to remember that the operator, as already mentioned herein above, upon requests by the user, may shift and activate selectively lamps 9, thus, varying the time and the areas of treatment.

On the basis of the description, it is evident that the solarium allows to better comply with the requirements of the user/client, achieving the mentioned objects.

1. A solarium (1) which comprises a support (2) on which presses a frame (3), said frame supporting at least a bed (4), said bed forming a shaped surface (5), a user (6) resting on a rail shaped surface, said frame also supporting a cover (7) disposed above said bed to cover said user, means generating ultraviolet rays disposed in said bed and in said cover being fed by a power group, characterized by the fact that said frame (3) is connected to said support (2) by connection means (11), said connection means forming at least one substantially horizontal axis of rotation (12), said bed and said cover rotating around said axis of rotation to define different positions of use.

2. The solarium according to claim 1, which comprises first handling means cooperating with said connection means (11) capable of rotating said bed (4) and said cover (7), around at least said axis of rotation (12) to dispose said solarium in different positions.

3. The solarium according to claim 2, wherein said first handling means comprises at least an electric motor provided with Kinematic means which cooperate mechanically with said connection means.

4. The solarium according to claim 1, wherein said means generating ultraviolet rays (8) are supported by guide means associated with said bed and said cover, said guide means forming at least one shifting direction (13), said shifting direction being essentially parallel to the longitudinal axis formed by said bed (4) and said cover (7).

5. The solarium according to claim 4, wherein said guide means comprise at least a rail (14), a carriage (15) coupled with said rail (14) in a smooth-running manner, said carriage (15) supporting said means generating ultraviolet rays (8).

6. The solarium according to claim 5, wherein said carriage is moved by a second handling means.

7. The solarium according to claim 6, wherein said second handling means comprises an electric motor provided with Kinematic means.

8. The solarium according to claim 1, wherein said power group comprises means for interruption capable of turning on and turning off selectively said means generating ultraviolet rays (8) to vary the areas of display to said rays.

9. The solarium according to claim 1, which comprises a control unit 1 cooperating with said means which generate ultraviolet rays and first and second handling means to control and vary the work configuration of the solarium (1).

10. The solarium according to claim 1, wherein said connection means (11) are a pin (11a) which rotates integral with said frame (3) and which forms at least one axis (12).
